SEQUENCE LISTING (5 sequences)

SEQ ID NO: 1 - RNA, 20 nt, Homo sapiens
  source 1..20: mol_type = other RNA
cctgagcccgggccgcgcag

SEQ ID NO: 2 - RNA, 19 nt, Homo sapiens
  source 1..19: mol_type = other RNA
aatggatttttggagcagg

SEQ ID NO: 3 - RNA, 19 nt, Homo sapiens
  source 1..19: mol_type = other RNA
tggatttttggatcaggga

SEQ ID NO: 4 - RNA, 22 nt, Homo sapiens
  source 1..22: mol_type = other RNA
catcttccagtacagtgttgga

SEQ ID NO: 5 - RNA, 22 nt, Homo sapiens
  source 1..22: mol_type = other RNA
tctggggatgaggacagtgtgt

(19) United States
(12) Patent Application Publication
(10) Pub. No.: US 2025/0115913 A1
(43) Pub. Date: Apr. 10, 2025

(54) AGENT AND PHARMACEUTICAL COMPOSITION FOR TREATING AND/OR PREVENTING JOINT DISEASE

(71) Applicant: TOKAI UNIVERSITY EDUCATIONAL SYSTEM, Tokyo (JP)

(72) Inventors: Miki KATO, Kanagawa (JP); Masato SATO, Kanagawa (JP)

(21) Appl. No.: 18/836,332

(22) PCT Filed: Oct. 12, 2022
(86) PCT No.: PCT/JP2022/038131

§ 371 Date: Oct. 29, 2024

(30) Foreign Application Priority Data
Feb. 7, 2022 (JP) 2022-017120

(51) Int. Cl. C12N 15/113; A61P 19/02; G01N 33/50

(52) U.S. Cl. CPC C12N 15/1136; A61P 19/02; G01N 33/5023; C12N 2310/141

(57) ABSTRACT

An object of the present invention is to provide a novel treatment agent and/or preventive agent for a joint disease. The present invention provides an agent for treating and/or preventing a joint disease, containing one or more miRNAs selected from the group consisting of hsa-miR-1199-5p, hsa-miR-1246, hsa-miR-1290, hsa-miR-141-5p, and hsa-miR-4700-5p as an active ingredient. In addition, the present invention provides a pharmaceutical composition for treating and/or preventing a joint disease, containing the agent for treating and/or preventing the joint disease.

### TECHNICAL FIELD

### Cross-Reference to Related Applications

The present application claims priority based on Japanese Patent Application No. 2022-017120 filed on Feb. 7, 2022, the entire disclosure of which is incorporated herein by reference. The present invention relates to an agent and a pharmaceutical composition for treating and/or preventing a joint disease.

### BACKGROUND ART

An exosome is a membrane-based endoplasmic reticulums having a diameter of 30 nm to 100 nm formed and released via an endocytosis pathway of a cell, and includes a nucleic acid (for example, microRNA and messenger RNA) and a protein derived from the original cell.

The exosome is known to be responsible for information transfer between cells. Specifically, an exosome released from a cell can be received by another cell and acts on a receptor present on the cell surface on a recipient side to cause signal transduction. Further, the exosome inclusions can be incorporated into a cell on the recipient side, and can perform gene transcription regulation or the like. For example, the microRNA (miRNA) included in the exosome can bind to a target messenger RNA (mnRNA) in the cell on the recipient side to suppress the expression of a target gene.

NPLs 1 and 2 below report exosome miRNA (ExosomalmiRNA) derived from a chondrocyte sheet. NPL 1 discloses that exosome miRNA may be involved in the paracrine effect. NPL 2 discloses that the exosome secreted by a cell sheet may control gene expression/translation by transmitting miRNA to the cells on the recipient side.

In addition, in recent years, an information transmission mechanism via the exosome has attracted attention in the medical field, and research for elucidating a physiological function of the exosome and technical development for applying the physiological function to treatment of diseases have also been performed. For example, PTL 1 below discloses an exosome derived from a mesenchymal stem cell in which a specific microRNA is highly expressed, and a treatment agent for a disease containing the exosome derived from the mesenchymal stem cell. NPL 3 below discloses that some upregulated miRNAs are identified in exosomes derived from human bone mesenchymal stem cells (hBMSCs) under chondrogenesis induction, and that these miRNAs may play an important role in exosomes derived from mesenchymal stem cells (MSCs) in cartilage regeneration and ultimately in the treatment of arthritis.

### CITATION LIST

### Patent Literature

PTL 1: JP2019-156739A

### Non-Patent Literature

NPL 1: Toyoda et al., “Analysis of miRNA containing exosome secreted from chondrocyte sheet”, Abstract of the 32nd Annual Research Meeting of the Japanese Orthopaedic Association (2017)
NPL 2: Toyoda et al., “Analysis of miRNA containing exosome secreted from chondrocyte sheet”, poster of the 32nd Annual Research Meeting of the Japanese Orthopaedic Association (2017)
NPL 3: Sun et al., Journal of Cellular Biochemistry, 2019; 120: 171-181

### SUMMARY OF INVENTION

### Technical Problem

A main object of the present invention is to provide a novel treatment agent and/or preventive agent for a joint disease.

### Solution to Problem

The present inventors have found that a miRNA suitable for a treatment agent and/or a preventive agent for a joint disease exists in an exosome inclusion obtained from a cell derived from a joint tissue, and have completed the present invention.

That is, the present invention provides an agent for treating and/or preventing a joint disease, containing a miRNA as an active ingredient.

In one embodiment, the miRNA may be at least one selected from the group consisting of hsa-miR-1199-5p, hsa-miR-1246, hsa-miR-1290, hsa-miR-141-5p, and hsa-miR-4700-5p.

In another embodiment, the miRNA may satisfy one or both of requirements (i) and (ii) below:
(i) a miRNA that suppresses an expression level of at least one factor selected from the group consisting of MMP-3, IL-1β, IL-6, TNF-α, MMP-13, ADAMTS5, and VEGFA in a case of being transfected into a human synovial sarcoma cell line as compared with a human synovial sarcoma cell line transfected with a Negative control miRNA mimic having no target gene,
(ii) a miRNA that suppresses the expression level of at least one factor selected from the group consisting of MNP-3 and RUNX2 in a case of being transfected into a Human Bone chondrosarcoma cell line, as compared with a Human Bone chondrosarcoma cell line transfected with a negative control miRNA mimic having no target gene.

In addition, the miRNA may be included in an exosome.

The exosome may be obtained from a cell derived from a joint tissue.

The cell derived from the joint tissue may be obtained from a cell culture derived from the joint tissue.

The treating and/or preventing the joint disease may be treating and/or preventing inflammation in a joint.

The treating and/or preventing the joint disease may be treating and/or preventing inflammation in a joint, and the active ingredient may be at least one selected from the group consisting of hsa-miR-1199-5p, hsa-miR-1246, and hsa-miR-4700-5p.

The treating and/or preventing the joint disease may be treating and/or preventing cartilage degeneration in a joint.

The treating and/or preventing the joint disease may be treating and/or preventing cartilage degeneration in a joint, and the active ingredient may be at least one selected from the group consisting of hsa-miR-1199-5p, hsa-miR-1246, hsa-miR-1290, and hsa-miR-4700-5p.

The treating and/or preventing cartilage degeneration in the joint may be treating and/or preventing cartilage damage in the joint.

The form of the agent may be an injection.

The present invention also provides a syringe in which the agent is filled in a syringe barrel.

The present invention also provides a pharmaceutical composition for treating and/or preventing a joint disease, containing the agent.

The present invention also provides a pharmaceutical composition for treating and/or preventing a joint disease, containing at least one precursor selected from the group consisting of a precursor of hsa-miR-1199-5p, a precursor of hsa-miR-1246, a precursor of hsa-miR-1290, a precursor of hsa-miR-141-5p, and a precursor of hsa-miR-4700-5p.

The present invention also provides a pharmaceutical composition for treating and/or preventing a joint disease, containing a vector expressing one or more miRNAs selected from the group consisting of hsa-miR-1199-5p, hsa-miR-1246, hsa-miR-1290, hsa-miR-141-5p and hsa-miR-4700-5p.

The form of the pharmaceutical composition may be the injection.

The present invention also provides a syringe in which a pharmaceutical composition is filled in a syringe barrel.

### Advantageous Effects of Invention

According to the present invention, a novel treatment agent and/or preventive agent for a joint disease is provided. The effects of the present invention are not limited to the effects described herein, and may be any of the effects described in the present description.

### BRIEF DESCRIPTION OF DRAWINGS

FIG. 1 is a view showing a procedure of miRNA analysis by a microarray.

FIG. 2 is a view showing a result of miRNA analysis by the microarray.

FIG. 3 is a view showing a result of a miRNA expression confirmation test by q-PCR.

FIG. 4 is a graph showing an expression level of MNP-3 gene in a Human Bone chondrosarcoma cell line SW1353.

FIG. 5 is a graph showing an expression level of MNP-3 gene in human synovial sarcoma cell line SW982.

FIG. 6 shows a result of small RNA-sequence analysis.

FIG. 7 is a graph showing the expression levels of various genes in the Human Bone chondrosarcoma cell line SW1353.

FIG. 8 is a graph showing the expression levels of various genes in the human synovial sarcoma cell line SW982.

### DESCRIPTION OF EMBODIMENTS

Hereinafter, preferred embodiments for performing the present invention will be described. The embodiments described below illustrate representative embodiments of the present invention, and the scope of the present invention is not limited only to these embodiments.

1. Agent for Treating and/or Preventing Joint Disease

An embodiment of the present invention is an agent for treating and/or preventing a joint disease, containing miRNA as an active ingredient. Examples of the miRNA that can be used as an active ingredient of the agent for treating and/or preventing a joint disease include a miRNA that suppresses the expression level of MMP-3 in a case of being transfected into a human synovial sarcoma cell line as compared with a human synovial sarcoma cell line transfected with a negative control miRNA mimic having no target gene. In one embodiment, examples of the miRNA that can be used as the active ingredient of the agent for treating and/or preventing a joint disease include one or more miRNAs selected from the group consisting of hsa-miR-1199-5p, hsa-miR-1246, hsa-miR-1290, hsa-miR-141-5p, and hsa-miR-4700-5p. In the present description, the miRNA contained as an active ingredient in the agent for treating and/or preventing a joint disease according to the present embodiment is also simply referred to as “miRNA of active ingredient”. Hereinafter, details of the present embodiment will be described.

### 1-1. Ingredient

miRBase (http://www.mirbase.org/) is an online database in which base sequences and annotations of miRNAs are stored. The base sequences, the accession numbers, and the sequence numbers of hsa-miR-1199-5p, hsa-miR-1246, hsa-miR-1290, hsa-miR-141-5p, and hsa-miR-4700-5p registered in miRBase are shown in Table 1 below. In the base sequences described in the present description, the left end is the 5′ end and the right end is the 3′ end unless otherwise specified.

TABLE 1

| miRNA | Base sequence | Accession number | Sequence number |
|---|---|---|---|
| hsa-miR-1199-5p | CCUGAGCCCGGGCCGCGCAG | MIMAT0031119 | 1 |
| hsa-miR-1246. | AAUGGAUUUUUGGAGCAGG | MIMAT0005898 | 2 |
| hsa-miR-1290 | UGGAUUUUUGGAUCAGGGA | MIMAT0005880 | 3 |
| hsa-miR-141-5p | CAUCUUCCAGUACAGUGUUGGA | MIMAT0004598 | 4 |
| hsa-miR-4700-5p | UCUGGGGAUGAGGACAGUGUGU | MIMAT0019796 | 5 |

In the present description, “hsa-miR-1199-5p” is a miRNA made of the base sequence of SEQ ID NO: 1 and/or a homolog thereof. “hsa-miR-1246” is a miRNA made of the base sequence of SEQ ID NO: 2 and/or a homolog thereof. “hsa-miR-1290” is a miRNA made of the base sequence of SEQ ID NO: 3 and/or a homolog thereof. “hsa-miR-141-5p” is a miRNA made of the base sequence of SEQ ID NO: 4 and/or a homolog thereof. “hsa-miR-4700-5p” is a miRNA made of the base sequence of SEQ ID NO: 5 and/or a homolog thereof.

The miRNA included in the agent for treating and/or preventing a joint disease according to the present embodiment is a mature miRNA (MaturemiRNA). A typical process for the production of mature miRNAs is as follows. A single-stranded primary transcript (PrimarymiRNA: pri-miRNA) having one or more hairpin structures is generated from the miRNA gene by RNA polymerase II. The pri-miRNA is cleaved by Drosha that is a ribonuclease III system enzyme, to produce a precursor miRNA (PrecursormiRNA: pre-miRNA) that is an intermediate precursor in a hairpin form. The pre-miRNA is cleaved in a cytoplasm by Dicer that is a ribonuclease III system enzyme, to produce a double-stranded miRNA including the mature miRNA and the miRNA* on the antisense side thereof. The double-stranded miRNA is incorporated into the RNA-induced silencing complex (RISC). The double-stranded miRNA incorporated into the RISC is released in the RISC to form two single-stranded miRNAs. Among them, one unstable single strand is degraded, and the other stable single strand functions as a mature miRNA.

In the agent for treating and/or preventing a joint disease according to an embodiment of the present invention, examples of the miRNA as an active ingredient include a miRNA satisfying one or both of requirements (i) and (ii) below:
(i) a miRNA that suppresses the expression level of at least one type of factors selected from the group consisting of MMP-3, IL-1β, IL-6, TNF-α, MMP-13, ADAMTS5, and VEGFA in a case of being transfected into a human synovial sarcoma cell line as compared with a human synovial sarcoma cell line transfected with a negative control miRNA mimic having no target gene,
(ii) a miRNA that suppresses the expression level of at least one type of factors selected from the group consisting of MMP-3 and RUNX2 in a case of being transfected into a Human Bone chondrosarcoma cell line, as compared with a Human Bone chondrosarcoma cell line transfected with a negative control miRNA mimic having no target gene.

Typically, the method for measuring the expression level of the factor can be performed by the method described in Examples of the present application. Therefore, in such an embodiment, in the measurement of the expression level of the factor, the SW982 line can be used as a human synovial sarcoma cell line. In addition, in the measurement of the expression level of the factor, the SW1353 line can be used as a Human Bone chondrosarcoma cell line. In addition, the measurement of the expression level of the factor is preferably performed in a state in which genes related to inflammation and cartilage degeneration are easily expressed by IL-1b stimulation as in Examples.

In the agent for treating and/or preventing a joint disease according to a preferred embodiment of the present invention, the miRNA as an active ingredient is one or more miRNAs selected from the group consisting of hsa-miR-1199-5p, hsa-miR-1246, hsa-miR-1290, hsa-miR-141-5p, and hsa-miR-4700-5p as described above. The miRNA as an active ingredient is preferably a combination of hsa-miR-1246 and/or hsa-miR-1290, hsa-miR-4700-5p or hsa-miR-1199-5p, hsa-miR-1246 and hsa-miR-1290. That is, the miRNA as an active ingredient is preferably hsa-miR-1246 singly, hsa-miR-1290 singly, hsa-miR-4700-5p singly, a combination of hsa-miR-1246 and hsa-miR-1290, or a combination of hsa-miR-1199-5p, hsa-miR-1246, and hsa-miR-1290. Thereby, more effective treatment and/or prevention of a joint disease can be expected.

The miRNA as an active ingredient may be, for example, produced in an organism, produced in a cell or a cell line collected from an organism, artificially synthesized, or commercially obtained.

The miRNA as an active ingredient is preferably an exosome miRNA (ExosomalmiRNA). In the present description, the “exosome miRNA” includes both the miRNA extracted from the exosome and the miRNA included in the exosome.

The miRNA as an active ingredient may be contained in the agent of the present embodiment together with a pharmaceutically acceptable carrier (for example, in a state of being included in a carrier or in a state of being in a complex with a carrier). The miRNA as an active ingredient may be included in, for example, a carrier, and is preferably included in an exosome.

In a case where the miRNA as an active ingredient is a miRNA extracted from an exosome or a miRNA included in an exosome (that is, an exosome miRNA), the exosome may be formed in a cell and released from the cell (that is, obtained from a cell), or may be artificially produced. The exosome is preferably obtained from a cell, more preferably obtained from cells derived from a joint tissue, and still more preferably obtained from chondrocytes. These cells are suitable for obtaining the miRNA as an active ingredient. The cells derived from the joint tissue are preferably obtained from a cell culture derived from the joint tissue. The chondrocytes are preferably obtained from a cell culture derived from cartilage tissue. These cell cultures are suitable for obtaining the miRNA as an active ingredient in an efficient manner or in a large amount. The cell culture may be obtained by, for example, two-dimensional culture (plane culture) or three-dimensional culture. The shape of the cell culture may be appropriately selected according to the type of cells, culture conditions, and the like, and may be, for example, a sheet-like shape, a granular shape, a fibrous shape (thread-like shape), or a net-like shape (mesh-like shape).

The cells derived from the joint tissue or the cells derived from the cartilage tissue may be, for example, those derived from joint tissue or cartilage tissue of an animal with polydactyly, those derived from joint tissue or cartilage tissue of an animal with hyperdactyly, or those derived from cartilage tissue (for example, knee cartilage tissue) of an animal. The animal is preferably a mammal, more preferably a primate animal, and still more preferably a human. The joint tissue or cartilage tissue may be collected from, for example, a tissue obtained at the time of resection of a surplus finger or surplus toe of a human. The cartilage tissue may be collected from waste tissue obtained at the time of artificial joint replacement of a human (adult).

The process of obtaining the exosome from the cell may be performed by a method known to those skilled in the art. For example, cells may be cultured in a liquid medium to release exosomes into the liquid medium, and then the exosomes may be recovered from the medium supernatant. In addition, it may be confirmed by a technique known to those skilled in the art that the exosome obtained from the cell includes the miRNA as an active ingredient.

The agent of the present embodiment can be obtained by formulating the miRNA as an active ingredient described above and, if necessary, the carrier (for example, exosome) by a technique known in the art. For formulation, the agent of the present embodiment may contain a pharmaceutically acceptable ingredient as long as the effect of the present invention is not impaired, in addition to the miRNA as an active ingredient and the carrier. The pharmaceutically acceptable ingredient may be appropriately selected by those skilled in the art from, for example, ingredients known in the art according to the application and form. Examples of the pharmaceutically acceptable ingredient include an excipient, a diluent, a suspension, a dispersant, a preservative, a stabilizer, and a buffer.

### 1-2. Form

The form of the agent according to the present embodiment may be a known form in the art, and may be, for example, oral formulations such as tablets, capsules, powders, granules, granules, granules, pills, suspensions, emulsions, solutions, and syrups, and non-oral formulations such as injections, drops, and external formulations. The form of the agent is preferably an injection. Thereby, the treatment and/or prevention of the joint disease can be performed minimally invasively and effectively. The injection may be, for example, in the form of a solution, a suspension, or an emulsion, or may be a solid formulation that is used by being dissolved at the time of use.

In a case where the agent of the present embodiment is an injection, the injection may be filled in a syringe barrel of a syringe. That is, the present invention can also provide a syringe in which the agent for treating and/or preventing a joint disease is filled in the syringe barrel.

In the present embodiment, a cell culture derived from the joint tissue described above and including the miRNA as an active ingredient may be used as an agent for treating and/or preventing a joint disease. In the present invention, the cell culture can also be referred to as a cell aggregate. In the present invention, the cell culture (cell aggregate) refers to a cell culture formed ex vivo or a cell culture extracted ex vivo. As described above, the cell culture includes those obtained by two-dimensional culture (plane culture) or three-dimensional culture. Therefore, examples of the cell culture (cell aggregate) include a flat (sheet) cell culture (cell aggregate), and a stereoscopic cell mass. The size of the cell culture (cell aggregate) is not limited, but in the case of a flat one, the area of the plane (one side) is preferably 1 to 1000 cm², more preferably 1 to 700 cm², and still more preferably 3 to 100 cm². In the case of a flat one, the thickness is also not limited, and is, for example, preferably 1 to 100 μm, more preferably 3 to 60 μm.

### 1-3. Target

In the present embodiment, the animal suffering from a joint disease to be treated and/or prevented is preferably a mammal, more preferably a primate animal, and still more preferably a human.

### 1-4. Application

The agent of the present embodiment is an agent for treating and/or preventing a joint disease. In the present description, the “joint disease” refers to a disease in which an abnormality such as deformation, damage, or inflammation occurs in a joint. The joint is a movable joint connecting the bone and the bone, and specifically includes cartilage, synovium, meniscus, ligament, and the like.

In the present embodiment, the treating and/or preventing the joint disease is preferably treating and/or preventing inflammation in a joint. That is, the agent of the present embodiment is preferably an agent for treating and/or preventing inflammation in a joint.

The agent for treating and/or preventing inflammation in the joint preferably contains at least one selected from the group consisting of hsa-miR-1199-5p, hsa-miR-1246, and hsa-miR-4700-5p as an active ingredient. That is, in the agent for treating and/or preventing inflammation in the joint, the active ingredient may be preferably hsa-miR-1199-5p singly, hsa-miR-1246 singly, hsa-miR-4700-5p singly, or a combination of hsa-miR-1199-5p and hsa-miR-1246. The results of the following examples support that agents containing such active ingredients are particularly suitable for treating and/or preventing inflammation in a joint.

In addition, the treating and/or preventing the joint disease is preferably treating and/or preventing cartilage degeneration in a joint. That is, the agent of the present embodiment is preferably an agent for treating and/or preventing cartilage degeneration in a joint. The treating and/or preventing cartilage degeneration in a joint is, for example, treating and/or preventing cartilage damage in the joint. That is, the agent of the present embodiment may be, for example, an agent for treating and/or preventing cartilage damage in a joint. In the present description, “cartilage damage” refers to a condition classified into grade 1 or more in Oueterbridge classification, and is one aspect of the cartilage degeneration.

The agent for treating and/or preventing cartilage degeneration (for example, cartilage damage) in a joint preferably contains hsa-miR-1246 and/or hsa-miR-1290, hsa-miR-4700-5p, or a combination of hsa-miR-1199-5p, hsa-miR-1246, and hsa-miR-1290 as an active ingredient. That is, in the agent for treating and/or preventing cartilage degeneration (for example, cartilage damage) in a joint, the active ingredient is preferably hsa-miR-1246 singly, hsa-miR-1290 singly, hsa-miR-4700-5p singly, a combination of hsa-miR-1246 and hsa-miR-1290, or a combination of hsa-miR-1199-5p, hsa-miR-1246, and hsa-miR-1290. The fact that the agent containing such an active ingredient is suitable for the above-described applications is supported by the results of the following Examples.

2. Pharmaceutical Composition Containing Agent for Treating and/or Preventing Joint Disease (First Pharmaceutical Composition)

The present invention also provides a pharmaceutical composition for treating and/or preventing a joint disease (hereinafter, also referred to as a “first pharmaceutical composition”), containing an agent for treating and/or preventing a joint disease as described above. In the first pharmaceutical composition according to an embodiment of the present invention, the agent for treating and/or preventing a joint disease is as described above in “1. Agent for treating and/or preventing joint disease”, and the explanation also applies to the present embodiment.

The first pharmaceutical composition according to the present embodiment may contain, in addition to the agent for treating and/or preventing the joint disease, one or combinations of two or more of other agents known in the art. The other agent may be, for example, another agent effective for treating and/or preventing a joint disease, and specific examples thereof include one or combinations of two or more selected from the group consisting of an agent for treating a joint disease, an agent for preventing a joint disease, an agent for treating inflammation, an agent for preventing inflammation, an agent for treating cartilage degeneration, an agent for preventing cartilage degeneration, an agent for treating cartilage damage, and an agent for preventing cartilage damage. In addition, the other agent may be, for example, an agent for complementing and/or reinforcing the effect of treating and/or preventing the joint disease.

The first pharmaceutical composition according to the present embodiment may be formulated by a technique known in the art. For formulation, in addition to the ingredients described above, a pharmaceutically acceptable ingredient may be contained as long as the effect of the present invention is not impaired. The pharmaceutically acceptable ingredient may be appropriately selected by those skilled in the art from, for example, ingredients known in the art according to the application and form. Examples of the pharmaceutically acceptable ingredient include an excipient, a diluent, a suspension, a dispersant, a preservative, a stabilizer, and a buffer.

The form of the first pharmaceutical composition according to the present embodiment may be the same as the form described in “1-2. Form” in “1. Agent for treating and/or preventing joint disease” above. That is, the form of the first pharmaceutical composition is preferably an injection. The injection may be filled in a syringe barrel of a syringe. That is, the present invention can also provide a syringe in which the first pharmaceutical composition is filled in the syringe barrel. In addition, similarly to the agent for treating and/or preventing a joint disease, a cell culture derived from a joint tissue containing a miRNA as an active ingredient can also be used as a pharmaceutical composition.

In addition, the target and application of the first pharmaceutical composition according to the present embodiment may be the same as the contents described in “1-3. Target” and “1-4. Application” in “1. Agent for treating and/or preventing joint disease” above.

3. Pharmaceutical Composition Containing Precursor of miRNA (Second Pharmaceutical Composition)

The present invention also provides a pharmaceutical composition for treating and/or preventing a joint disease (hereinafter, also referred to as a “second pharmaceutical composition”), containing a precursor of the “miRNA as an active ingredient”, for example, a pharmaceutical composition for treating and/or preventing a joint disease, containing one or more precursors selected from the group consisting of a precursor of hsa-miR-1199-5p, a precursor of hsa-miR-1246, a precursor of hsa-miR-1290, a precursor of hsa-miR-141-5p, and a precursor of hsa-miR-4700-5p.

The second pharmaceutical composition according to an embodiment of the present invention preferably contains a precursor of hsa-miR-1246 and/or a precursor of hsa-miR-1290, a precursor of hsa-miR-4700-5p, or a combination of a precursor of hsa-miR-1199-5p, a precursor of hsa-miR-1246, and a precursor of hsa-miR-1290. That is, the second pharmaceutical composition according to the present embodiment preferably contains the precursor of hsa-miR-1246 singly, the precursor of hsa-miR-1290 singly, the precursor of hsa-miR-4700-5p singly, a combination of the precursor of hsa-miR-1246 and the precursor of hsa-miR-1290, or a combination of the precursor of hsa-miR-1199-5p, the precursor of hsa-miR-1246, and the precursor of hsa-miR-1290. Thereby, more effective treatment and/or prevention of a joint disease can be expected.

In the present description, a “precursor” is an RNA capable of producing a mature miRNA by cleavage or double-stranded cleavage. The precursor contained in the second pharmaceutical composition is preferably one or more precursors selected from the group consisting of pri-miRNA, pre-miRNA, and double-stranded miRNA (hereinafter, also simply referred to as a “double-stranded miRNA”) made of a mature miRNA and an antisense strand thereof. That is, the second pharmaceutical composition according to the present embodiment preferably contains one or more precursors selected from the group consisting of pri-miRNA, pre-miRNA, and double-stranded miRNA of hsa-miR-1199-5p, pri-miRNA, pre-miRNA, and double-stranded miRNA of hsa-miR-1246, pri-miRNA, pre-miRNA, and double-stranded miRNA of hsa-miR-1290, pri-miRNA, pre-miRNA, and double-stranded miRNA of hsa-miR-141-5p, and pri-miRNA, pre-miRNA, and double-stranded miRNA of hsa-miR-4700-5p.

The second pharmaceutical composition more preferably contains any one of the following (A) to (F).
(A) One or more precursors selected from the group consisting of pri-miRNA, pre-miRNA, and double-stranded miRNA of hsa-miR-1246.
(B) One or more precursors selected from the group consisting of pri-miRNA, pre-miRNA, and double-stranded miRNA of hsa-miR-1290.
(C) A combination of one or more precursors (c-1) selected from the group consisting of pri-miRNA, pre-miRNA, and double-stranded miRNA of hsa-miR-1246 and one or more precursors (c-2) selected from the group consisting of pri-miRNA, pre-miRNA, and double-stranded miRNA of hsa-miR-1290.
(D) A combination of one or more precursors (d-1) selected from the group consisting of pri-miRNA, pre-miRNA, and double-stranded miRNA of hsa-miR-1199-5p, one or more precursors (d-2) selected from the group consisting of pri-miRNA, pre-miRNA, and double-stranded miRNA of hsa-miR-1246, and one or more precursors (d-3) selected from the group consisting of pri-miRNA, pre-miRNA, and double-stranded miRNA of hsa-miR-1290.
(E) One or more precursors selected from the group consisting of pri-miRNA, pre-miRNA, and double-stranded miRNA of hsa-miR-141-5p.
(F) One or more precursors selected from the group consisting of pri-miRNA, pre-miRNA, and double-stranded miRNA of hsa-miR-4700-5p.

The second pharmaceutical composition according to the present embodiment contains one or more precursors described above, thereby allowing to produce one or more miRNAs corresponding to the precursors. For example, the second pharmaceutical composition can produce one or more miRNAs selected from the group consisting of hsa-miR-1199-5p, hsa-miR-1246, hsa-miR-1290, hsa-miR-141-5p, and hsa-miR-4700-5p in a body of the animal suffering from a joint disease to be treated and/or prevented by being administered to the animal. One or more of the produced miRNAs can function for treating and/or preventing a joint disease in the body of the animal as described in “1. Agent for treating and/or preventing joint disease” above.

The precursor of hsa-miR-1199-5p, the precursor of hsa-miR-1246, the precursor of hsa-miR-1290, the precursor of hsa-miR-141-5p, and hsa-miR-4700-5p may be, for example, those produced in an organism, those produced in a cell or a cell line collected from an organism, those artificially synthesized, those commercially obtained, or the like.

The precursor of hsa-miR-1199-5p, the precursor of hsa-miR-1246, the precursor of hsa-miR-1290, the precursor of hsa-miR-141-5p, and hsa-miR-4700-5p may be contained in the second pharmaceutical composition according to the present embodiment together with a pharmaceutically acceptable carrier (for example, in a state of being contained in a carrier or in a state of being in a complex with a carrier).

The second pharmaceutical composition according to the present embodiment may contain, in addition to one or more of the precursors and, if necessary, the carrier, one or combinations of two or more of agents known in the art. The agent may be, for example, an agent effective for treating and/or preventing a joint disease, and specific examples thereof include one or combinations of two or more selected from the group consisting of an agent for treating a joint disease, an agent for preventing a joint disease, an agent for treating inflammation, an agent for preventing inflammation, an agent for treating cartilage degeneration, an agent for preventing cartilage degeneration, an agent for treating cartilage damage, and an agent for preventing cartilage damage. In addition, the agent may be, for example, an agent for complementing and/or reinforcing the effect of treating and/or preventing the joint disease.

The second pharmaceutical composition according to the present embodiment may be formulated by a technique known in the art. For formulation, in addition to the ingredients described above, a pharmaceutically acceptable ingredient may be contained as long as the effect of the present invention is not impaired. The pharmaceutically acceptable ingredient may be appropriately selected by those skilled in the art from, for example, ingredients known in the art according to the application and form. Examples of the pharmaceutically acceptable ingredient include an excipient, a diluent, a suspension, a dispersant, a preservative, a stabilizer, and a buffer.

The form of the second pharmaceutical composition according to the present embodiment may be the same as the form described in “1-2. Form” in “1. Agent for treating and/or preventing joint disease” above. That is, the form of the second pharmaceutical composition is preferably an injection. The injection may be filled in a syringe barrel of a syringe. That is, the present invention can also provide a syringe in which the second pharmaceutical composition is filled in the syringe barrel.

In addition, the target and application of the second pharmaceutical composition according to the present embodiment may be the same as the contents described in “1-3. Target” and “1-4. Application” in “1. Agent for treating and/or preventing joint disease” above.

In the application of the present embodiment, in a case where the treating and/or preventing a joint disease is the treating and/or preventing inflammation in a joint, that is, in a case where the second pharmaceutical composition is a pharmaceutical composition for treating and/or preventing inflammation in a joint, the second pharmaceutical composition preferably contains at least one selected from the group consisting of a precursor of hsa-miR-1199-5p, a precursor of hsa-miR-1246, and a precursor of hsa-miR-4700-5p. The precursor contained in the second pharmaceutical composition for treating and/or preventing inflammation in a joint is preferably a precursor of hsa-miR-1199-5p singly, a precursor of hsa-miR-1246 singly, a precursor of hsa-miR-4700-5p singly, or a combination of a precursor of hsa-miR-1199-5p and a precursor of hsa-miR-1246. These precursors are particularly suitable for treating and/or preventing inflammation in a joint.

The second pharmaceutical composition for treating and/or preventing inflammation in a joint more preferably contains any one of the following (A) to (D).
(A) One or more precursors selected from the group consisting of pri-miRNA, pre-miRNA, and double-stranded miRNA of hsa-miR-1199-5p.
(B) One or more precursors selected from the group consisting of pri-miRNA, pre-miRNA, and double-stranded miRNA of hsa-miR-1246.
(C) A combination of one or more precursors (c-1) selected from the group consisting of pri-miRNA, pre-miRNA, and double-stranded miRNA of hsa-miR-1199-5p and one or more precursors (c-2) selected from the group consisting of pri-miRNA, pre-miRNA, and double-stranded miRNA of hsa-miR-1246.
(D) One or more precursors selected from the group consisting of pri-miRNA, pre-miRNA, and double-stranded miRNA of hsa-miR-4700-5p.

In the application of the present embodiment, in a case where the treating and/or preventing a joint disease is treating and/or preventing cartilage degeneration (for example, cartilage damage) in a joint, that is, in a case where the second pharmaceutical composition is a pharmaceutical composition for treating and/or preventing cartilage degeneration (for example, cartilage damage) in a joint, the second pharmaceutical composition preferably contains at least one selected from the group consisting of a precursor of hsa-miR-1246, a precursor of hsa-miR-1290, and a precursor of hsa-miR-4700-5p, or a combination of a precursor of hsa-miR-1199-5p, a precursor of hsa-miR-1246, and a precursor of hsa-miR-1290. The precursor included in the second pharmaceutical composition for treating and/or preventing cartilage degeneration (for example, cartilage damage) in a joint is preferably a precursor of hsa-miR-1246 singly, a precursor of hsa-miR-1290 singly, a precursor of hsa-miR-4700-5p singly, a combination of a precursor of hsa-miR-1246 and a precursor of hsa-miR-1290, or a combination of a precursor of hsa-miR-1199-5p, a precursor of hsa-miR-1246, and a precursor of hsa-miR-1290. These precursors are particularly suitable for treating and/or preventing cartilage degeneration (for example, cartilage damage) in a joint.

The second pharmaceutical composition for treating and/or preventing cartilage degeneration (for example, cartilage damage) in a joint more preferably contains any one of the following (A) to (E).
(A) One or more precursors selected from the group consisting of pri-miRNA, pre-miRNA, and double-stranded miRNA of hsa-miR-1246.
(B) One or more precursors selected from the group consisting of pri-miRNA, pre-miRNA, and double-stranded miRNA of hsa-miR-1290.
(C) A combination of one or more precursors (c-1) selected from the group consisting of pri-miRNA, pre-miRNA, and double-stranded miRNA of hsa-miR-1246 and one or more precursors (c-2) selected from the group consisting of pri-miRNA, pre-miRNA, and double-stranded miRNA of hsa-miR-1290.
(D) A combination of one or more precursors (d-1) selected from the group consisting of pri-miRNA, pre-miRNA, and double-stranded miRNA of hsa-miR-1199-5p, one or more precursors (d-2) selected from the group consisting of pri-miRNA, pre-miRNA, and double-stranded miRNA of hsa-miR-1246, and one or more precursors (d-3) selected from the group consisting of pri-miRNA, pre-miRNA, and double-stranded miRNA of hsa-miR-1290.
(E) One or more precursors selected from the group consisting of pri-miRNA, pre-miRNA, and double-stranded miRNA of hsa-miR-4700-5p.

### 4. Pharmaceutical Composition Containing Vector (Third Pharmaceutical Composition)

The present invention also provides a pharmaceutical composition for treating and/or preventing a joint disease, containing a vector expressing the “miRNA of an active ingredient” (hereinafter, also referred to as a “third pharmaceutical composition”), for example, a pharmaceutical composition for treating and/or preventing a joint disease, containing a vector expressing at least one miRNA selected from the group consisting of hsa-miR-1199-5p, hsa-miR-1246, hsa-miR-1290, hsa-miR-141-5p, and hsa-miR-4700-5p.

The third pharmaceutical composition according to an embodiment of the present invention preferably contains a vector expressing at least one selected from the group consisting of hsa-miR-1246, hsa-miR-1290, and hsa-miR-4700-5p, or a vector expressing hsa-miR-1199-5p, hsa-miR-1246, and hsa-miR-1290. The third pharmaceutical composition according to the present embodiment preferably contains a vector expressing hsa-miR-1246, a vector expressing hsa-miR-1290, a vector expressing hsa-miR-1246 and hsa-miR-1290, a vector expressing hsa-miR-4700-5p, or a vector expressing hsa-miR-1199-5p, hsa-miR-1246, and hsa-miR-1290. Thereby, more effective treatment and/or prevention of a joint disease can be expected.

The vector contained in the third pharmaceutical composition according to the present embodiment can be obtained by inserting a polynucleotide encoding one or more miRNAs selected from the group consisting of hsa-miR-1199-5p, hsa-miR-1246, hsa-miR-1290, hsa-miR-141-5p, and hsa-miR-4700-5p into an appropriate vector appropriately selected by those skilled in the art. The polynucleotide encoding hsa-miR-1199-5p, the polynucleotide encoding hsa-miR-1246, the polynucleotide encoding hsa-miR-1290, the polynucleotide encoding hsa-miR-141-5p, and the polynucleotide encoding hsa-miR-4700-5p may be inserted into the same vector or may be inserted into different vectors. The vector contained in the third pharmaceutical composition may be a non-viral vector or a viral vector.

The third pharmaceutical composition according to the present embodiment can express one or more miRNAs corresponding to the vector by containing the vector described above. For example, the third pharmaceutical composition can express one or more miRNAs selected from the group consisting of hsa-miR-1199-5p, hsa-miR-1246, hsa-miR-1290, hsa-miR-141-5p, and hsa-miR-4700-5p in the body of an animal suffering from a joint disease to be treated and/or prevented by being administered to the animal. One or more of the expressed miRNAs can function for treating and/or preventing a joint disease in the body of the animal as described in “1. Agent for treating and/or preventing joint disease” above.

The third pharmaceutical composition according to the present embodiment may contain, in addition to the vector, one or combinations of two or more of the agents known in the art. The agent may be, for example, an agent effective for treating and/or preventing a joint disease, and specific examples thereof include one or combinations of two or more selected from the group consisting of an agent for treating a joint disease, an agent for preventing a joint disease, an agent for treating inflammation, an agent for preventing inflammation, an agent for treating cartilage degeneration, an agent for preventing cartilage degeneration, an agent for treating cartilage damage, and an agent for preventing cartilage damage. In addition, the agent may be, for example, an agent for complementing and/or reinforcing the effect of treating and/or preventing the joint disease.

The third pharmaceutical composition according to the present embodiment may be formulated by a technique known in the art. For formulation, in addition to the ingredients described above, a pharmaceutically acceptable ingredient may be contained as long as the effect of the present invention is not impaired. The pharmaceutically acceptable ingredient may be appropriately selected by those skilled in the art from, for example, ingredients known in the art according to the application and form. Examples of the pharmaceutically acceptable ingredient include an excipient, a diluent, a suspension, a dispersant, a preservative, a stabilizer, and a buffer.

The form of the third pharmaceutical composition according to the present embodiment may be the same as the form described in “1-2. Form” in “1. Agent for treating and/or preventing joint disease” above. That is, the form of the third pharmaceutical composition is preferably an injection. The injection may be filled in a syringe barrel of a syringe. That is, the present invention can also provide a syringe in which the third pharmaceutical composition is filled in a syringe barrel.

In addition, the target and application of the third pharmaceutical composition according to the present embodiment may be the same as the contents described in “1-3. Target” and “1-4. Application” in “1. Agent for treating and/or preventing joint disease” above.

In the application of the present embodiment, in a case where the treating and/or preventing a joint disease is the treating and/or preventing inflammation in a joint, that is, in a case where the third pharmaceutical composition is a pharmaceutical composition for treating and/or preventing inflammation in a joint, the third pharmaceutical composition preferably contains the vector expressing at least one selected from the group consisting of hsa-miR-1199-5p, hsa-miR-1246, and hsa-miR-4700-5p. The third pharmaceutical composition for treating and/or preventing inflammation in a joint preferably contains a vector expressing hsa-miR-1199-5p, a vector expressing hsa-miR-1246, a vector expressing hsa-miR-4700-5p, or a vector expressing hsa-miR-1199-5p and hsa-miR-1246. These vectors are particularly suitable for treating and/or preventing inflammation in a joint.

In the application of the present embodiment, in a case where the treating and/or preventing a joint disease is treating and/or preventing cartilage degeneration (for example, cartilage damage) in a joint, that is, in a case where the third pharmaceutical composition is a pharmaceutical composition for treating and/or preventing cartilage degeneration (for example, cartilage damage) in a joint, the third pharmaceutical composition preferably contains a vector expressing at least one selected from the group consisting of hsa-miR-1246, hsa-miR-1290, and hsa-miR-4700-5p, or a vector expressing hsa-miR-1199-5p, hsa-miR-1246, and hsa-miR-1290. That is, the third pharmaceutical composition for treating and/or preventing cartilage degeneration (for example, cartilage damage) in a joint preferably contains a vector expressing hsa-miR-1246, a vector expressing hsa-miR-1290, a vector expressing hsa-miR-1246 and hsa-miR-1290, a vector expressing hsa-miR-4700-5p, or a vector expressing hsa-miR-1199-5p, hsa-miR-1246, and hsa-miR-1290. These vectors are particularly suitable for treating and/or preventing cartilage degeneration (for example, cartilage damage) in a joint.

### 5. Determination Method and Marker

As described above, the “miRNA as an active ingredient” is effective for treating and/or preventing a joint disease. Therefore, in order to determine whether or not a cell collected from a certain donor (including a cell aggregate constructed from the cell) is effective for treating and/or preventing a joint disease, the “miRNA as an active ingredient” can be used as an index. Therefore, in a preferred embodiment, the present invention provides a method for determining whether or not it is effective for treating and/or preventing a joint disease using, as an index, a miRNA in an exosome released from a cell derived from a joint tissue to be targeted, in which
the miRNA is at least one selected from the group consisting of hsa-miR-1199-5p, hsa-miR-1246, hsa-miR-1290, hsa-miR-141-5p, and hsa-miR-4700-5p. In addition, the present invention provides a method for determining whether or not it is effective for treating and/or preventing a joint disease using, as an index, a miRNA in an exosome released from a cell derived from a joint tissue to be targeted, in which
the miRNA satisfies one or both of requirements (i) and (ii) below:
(i) A miRNA that suppresses the expression level of at least one of factors selected from the group consisting of MMP-3, IL-1β, IL-6, TNF-α, MMP-13, ADAMTS5, and VEGFA in a case of being transfected into a human synovial sarcoma cell line as compared with a human synovial sarcoma cell line transfected with a negative control miRNA mimic having no target gene.
(ii) A miRNA that suppresses the expression level of at least one of factors selected from the group consisting of MMP-3 and RUNX2 in a case of being transfected into a Human Bone chondrosarcoma cell line, as compared with a Human Bone chondrosarcoma cell line transfected with a negative control miRNA mimic having no target gene.

In the present embodiment, typically, in a case where an amount of the “miRNA as an active ingredient” in the exosome released from the cell derived from the joint tissue to be targeted is equal to or more than a preset reference value, it can be determined whether or not the cell derived from the joint tissue to be targeted are effective for treating and/or preventing the joint disease. The method for measuring the amount of the “miRNA as an active ingredient” is not limited, and examples thereof include q-PCR, ELISA method, miRNA microarray, and RNA sequence. As the reference value, for example, the cell derived from a joint tissue, being already known to be effective for treating and/or preventing a joint disease is used to measure the amount of “miRNA as an active ingredient” in the exosome released from the cells, thereby allowing to be used as the reference value. In addition, a criterion such as several times or more (for example, 1 time or more, 2 times or more, 3 times or more, or the like) compared to the signal of the exosome of the cell having clear effectiveness can be determined previously. In the present embodiment, the term “cell derived from a joint tissue” also includes cell aggregates constructed in vitro or in vivo from a cell derived from a joint tissue. As described above, the “miRNA as an active ingredient” can be used as a marker for determining whether or not cells collected from a certain donor are effective for treating and/or preventing a joint disease. Therefore, the present invention provides a marker for determining whether or not the cell aggregate is effective for treating and/or preventing a joint disease, the marker being made of the “miRNA as an active ingredient”. Specifically, the present invention provides a marker for determining whether or not the cell aggregate is effective for treating and/or preventing a joint disease, the marker being made of at least one miRNA selected from the group consisting of hsa-miR-1199-5p, hsa-miR-1246, hsa-miR-1290, hsa-miR-141-5p, and hsa-miR-4700-5p. In addition, in another embodiment, the present invention provides a marker for determining whether or not the cell aggregate is effective for treating and/or preventing a joint disease, the marker being made of miRNA, in which the miRNA satisfies one or both of requirements (i) and (ii) below:
(i) A miRNA that suppresses the expression level of at least one of factors selected from the group consisting of MMP-3, IL-1β, IL-6, TNF-α, MMP-13, ADAMTS5, and VEGFA in a case of being transfected into a human synovial sarcoma cell line as compared with a human synovial sarcoma cell line transfected with a negative control miRNA mimic having no target gene.
(ii) A miRNA that suppresses the expression level of at least one of factors selected from the group consisting of MMP-3 and RUNX2 in a case of being transfected into a Human Bone chondrosarcoma cell line, as compared with a Human Bone chondrosarcoma cell line transfected with a negative control miRNA mimic having no target gene. The marker of the present invention can be used in the above determination method.

### 6. Method for Producing Exosome

In another embodiment, the present invention provides a method for producing an exosome containing a “miRNA as an active ingredient”, the method including a step of culturing a cell containing the “miRNA as an active ingredient”. Preferably, the present invention provides a method for producing an exosome containing at least one miRNA selected from the group consisting of hsa-miR-1199-5p, hsa-miR-1246, hsa-miR-1290, hsa-miR-141-5p, and hsa-miR-4700-5p, the method including a step of culturing a cell containing at least one miRNA selected from the group consisting of hsa-miR-1199-5p, hsa-miR-1246, hsa-miR-1290, hsa-miR-141-5p, and hsa-miR-4700-5p. Examples of the cell include a cell derived from a joint tissue (more preferably chondrocytes). A human cell is preferable. The culture method, details of the “miRNA as an active ingredient”, and the like are the same as those described above (for example, those described in “1. Agent for treating and/or preventing joint disease” and “1-1. Ingredient”). In the embodiment, the method for producing an exosome of the present invention may further include a step of collecting the exosome obtained in the culturing step. As a method for collecting the exosome, a known method can be appropriately adopted.

### EXAMPLES

Hereinafter, the present invention will be described in more detail with reference to Examples, but the present invention is not limited to these Examples.

### Example 1

### (1-1) Cell Sheet Culture

Chondrocytes were isolated from the waste tissue during polydactyly surgery to prepare a first passage cell stock. Cell stocks were thawed and a second passage cells were seeded into a temperature-responsive culture insert (UpCell (registered trademark) insert, manufactured by CellSeed Inc.) at a density of 1×10⁴/cm². The medium was replaced every 3 to 4 days, and a cell sheet (PD sheet) that had been cultured for 2 weeks and the completed cell sheet was used for the experiment. A DMEM-F12 medium containing 20% FBS and 1% anti-biotics was used at the time of seeding the primary culture cells, and a DMEM-F12 medium containing 20% FBS, 1% anti-biotics, and 100 μg/mL ascorbic acid was used for subsequent culture.

### (1-2) Collection of Cell Sheet Culture Supernatant

In order to collect the exosome released from the cell sheet, the culture supernatant was collected at the timing after a lapse of 72 hours from the 11th day from the start of culture. Centrifugation was performed under conditions of 2000 g, 10 minutes, and 4° C., and cell debris and the like were removed through a 0.22 μm filter. A sample was stored at −80° C.

The samples used in a property confirmation experiment (confirmation of exosome marker by colloidal gold immunoelectron microscope, particle count by nanoparticle analysis system, and confirmation by intracellular tracking of exosome) of the exosome released into the cell sheet culture supernatant were cultured in a serum-free medium for 72 hours after completion of the sheet.

### (1-3) Confirmation of Exosome Marker by Colloidal Gold Immunoelectron Microscope

Expression of a surface marker of the exosome was confirmed by the following procedure.
a. An exosome was extracted using ExoQuick-TC according to protocol.
b. Exosome specific primary antibody (CD63, CD81) was used as a primary antibody, and Anti-Rabbit IgG (whole molecule)-Gold was used as a secondary antibody.
c. The exosome was immobilized with 4% paraformaldehyde, and the immobilized exosome solution was placed on a carbon-Formvar coated 200 mesh nickel grid.
d. The mesh was immersed in Blocking Buffer (0.4% BSA in PBS).
e. The mesh was sequentially immersed in 1st Antibody in Blocking Buffer and 2nd Anti-body in 2nd Ab Dilution.
f. Negative stain was performed with uranylacetate.
g. The mesh was dried and observed with an electron microscope.

As a result, expression of exosome markers CD63 and CD81 was confirmed. That is, it was confirmed that the cell sheet released the exosome into a culture solution.

(1-4) Particle Count with Nanoparticle Tracking Analysis System
a. An exosome was extracted using ExoQuick-TC according to protocol.
b. Exosome particles moved by Brownian motion in the solution were photographed by a laser light scattering method. For photographing, an LM10 NanoSight instrument was used.
c. The particles were measured three times, and the average particle diameter, mode diameter, and concentration of the exosome particles were calculated.

As a result, it was confirmed that the membrane-based endoplasmic reticulum (exosome) released from the cell sheet into the culture solution was an endoplasmic reticulum generally known to have a diameter of 100 nm.

### (1-5) Confirmation by Intracellular Tracking of Exosome

a. An exosome was extracted using ExoQuick-TC according to protocol.
b. The extracted exosome was stained with PKH67 Green Fluorescent Cell Linker Kit, and then unreacted dye was removed using Exosome Spin Columns.
c. Chondrocytes or human bone marrow-derived mesenchymal stem cells (hBM-MSCs) were seeded on 8 well chamber slides at 1×10⁴ cells/well on the day before the experiment, and labeled exosomes were added to the cells.
d. After incubation at 37° C. for 3 hours in the dark, the medium was removed, and 4% PFA was added to fix the cells.
e. Cytoskeletons were stained with Alexa Fluor 594 Phalloidin Conjugate, samples were encapsulated with DAPI-containing encapsulants and observed under fluorescence microscopy.

As a result, it was confirmed that the exosome released by the cell sheet was incorporated into chondrocytes and hBM-MSCs. In a case where the exosome is administered into a joint, it is considered that the exosome is incorporated into these cells and acts thereon.

(1-6) Microarray Analysis of miRNA in Polydactyly Chondrocyte Sheet and Exosome miRNA in Culture Supernatant

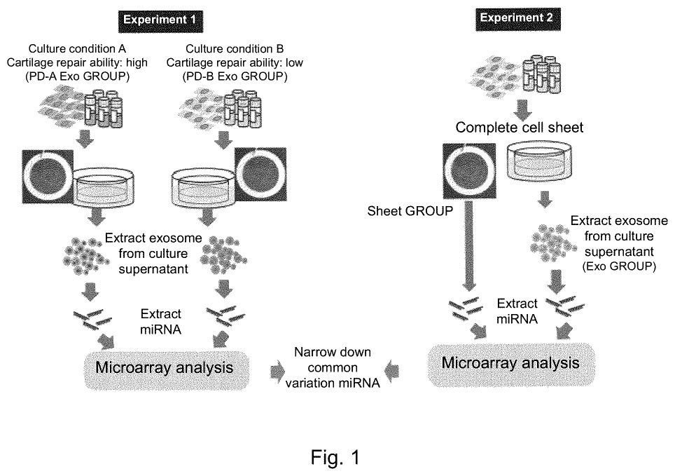

According to the procedure shown in FIG. 1 and the following, microarray analysis of the miRNA in the polydactyly chondrocyte sheet and the exosome miRNA (miRNA contained in the exosome) in the culture supernatant was performed.
a. Two types of cell sheets (PD-A Exo group and PD-B Exo group) were prepared from chondrocytes derived from patients with polydactyly from three donors (n=4) under different culture conditions (culture conditions A and B). It has been previously confirmed that cell sheets prepared under these two types of culture conditions have greatly different cartilage tissue repair abilities in an in vivo experiment using a heterologous orthotopic model animal. The miRNAs contained in the exosomes in these cell sheet culture supernatants were extracted using exoRNeasy Serum/Plasma Maxi Kit, and the miRNAs significantly varying under the culture condition A with excellent cartilage repair ability were narrowed down by microarray analysis (p<0.05, Fold Change >2). This was defined as “Experiment 1”.
b. miRNAs contained in cell sheets prepared from chondrocytes derived from patients with polydactyly from three donors (n=7) (Sheet group) and miRNAs contained in the exosome in the culture supernatant (Exosome group) were each extracted using RNeasy Mini Kit or exoRNeasy Serum/Plasma Maxi Kit, and miRNAs significantly varying in the exosome in the culture supernatant were narrowed down by microarray analysis (p<0.05, FoldChange >2). This was defined as “Experiment 2”.
c. For microarray analysis in Experiments 1 and 2, Agilent Human miRNA microarray (2549probe) was used. After the microarray analysis, common variation miRNAs were extracted among the miRNAs narrowed down in each of Experiments 1 and 2.
d. In order to subtract the influence of exosomes derived from serum, DMEM-F12 medium containing 20% FBS and 1% Anti-Biotics was used as a control sample.

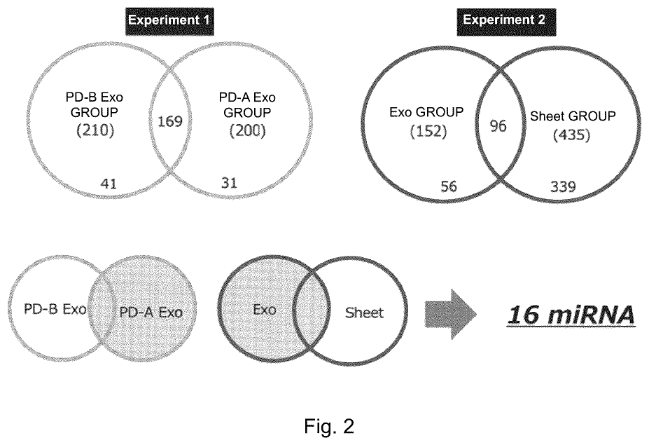

As a result, 152 miRNAs were significantly packaged in the exosome secreted from the cell sheet, and 200 miRNAs were significantly expressed under the culture condition A (PD-AExo group) having excellent cartilage repair ability (FIG. 2). 16 pieces of expression variation miRNAs common to both were extracted (FIG. 2).

(1-7) Confirmation of miRNA Expression by q-PCR

For the miRNAs narrowed down in the above Experiment (1-6), a validation test by q-PCR was performed. A correlation with the result of the microarray was confirmed for each probe, and miRNAs having a correlation in both the Experiments 1 and 2 were identified (correlation coefficient R>0.5). As an internal control, hsa-miR-6734-5p having the least variation in the sample in the microarray data was used.

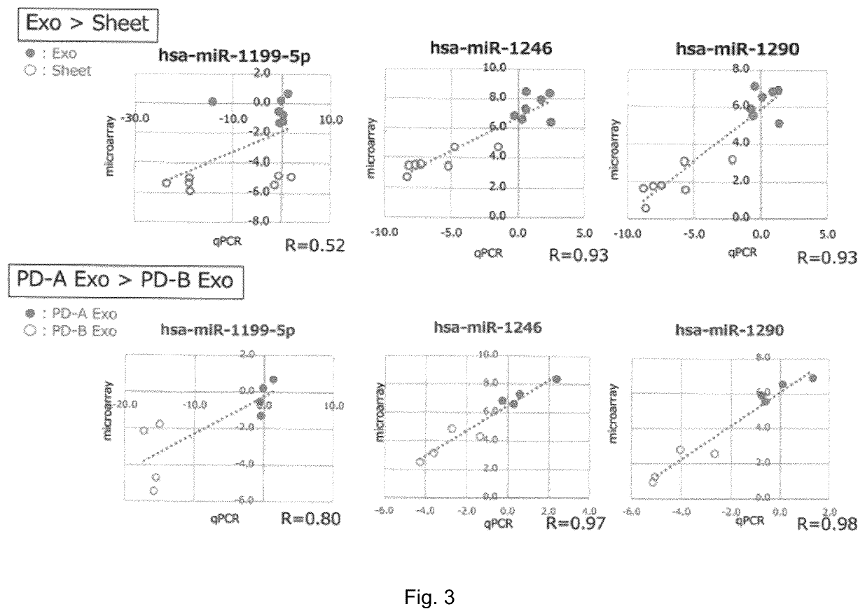

As a result, among the 16 miRNAs, 7 miRNAs were confirmed to have a 2-fold or more variation by PCR in at least one of Experiments 1 and 2. In both Experiments 1 and 2, there were 3 miRNAs in which a positive correlation with a correlation coefficient of 0.5 or more was observed between the signal value of the array and the −ΔCT value of PCR (Table 2 and FIG. 3). Specifically, the 3 miRNAs were hsa-miR-1199-5p, hsa-miR-1246, and hsa-miR-1290. In FIG. 3, the vertical axis of the graph represents the signal value of the microarray, and the horizontal axis represents the −ΔCT value of PCR.

TABLE 2

| | | | | | PD-B Exo vs PD-A Exo (n = 4) | | | |
|---|---|---|---|---|---|---|---|---|
| | PD vs Exo (n = 7) | | | | PD-A | PD-B | | |
| | PD −ΔCT | Exo −ΔCT | Log FC | Corr. | Exo −ΔCT | Exo −ΔCT | Log FC | Corr. |
| | average *1 | average *2 | *3 | *4 | average *5 | average *6 | *7 | *4 |
| hsa-miR-1199-59 | −11.4 | −1.9 | 9.4 | 0.52 | 0.0 | −15.9 | 15.9 | 0.80 |
| hsa-miR-1246 | −6.1 | 1.1 | 7.2 | 0.93 | 0.7 | −3.0 | 3.7 | 0.97 |
| hsa-miR-1290 | −6.6 | 0.3 | 6.9 | 0.93 | 0.0 | −4.2 | 4.2 | 0.98 |

*1 to *7 in Table 2 above mean the following.
*1: PCR data of PD group (average −ΔCT value)
*2: PCR data of Exo group (average −ΔCT value)
*3: Multiple number of difference in expression between PD group and Exo group (base 2 logarithm)
*4: Correlation with result of microarray (R is correlation coefficient)
*5: PCR data of PD-A Exo group (average −ΔCT value)
*6: PCR data of PD-B Exo group (average −ΔCT value)
*7: Multiple number of difference in expression between PD-A Exo group and PD-B Exo group (base 2 logarithm)

(1-8) Verification of Silencing of miRNAs Against Cartilage Catabolic Gene and Inflammation-Associated Gene

The functions of the 3 miRNAs identified in (1-7) above on cartilage catabolic genes and inflammation-associated genes in the target cell were investigated. In the present description, the “cartilage catabolic gene” means a gene involved in induction of cartilage degeneration.
a. Human Bone chondrosarcoma cell line SW1353 (n=4) or Human Synovial sarcoma cell line SW982 (n=4) was transfected with each miRNA singly or in combination of 3 types of miRNAs.
b. 24 hours after cell seeding, hsa-miR-1199-5p, 1246, 1290mimic (mirVana miRNA Mimics) was transfected using Lipofectamine RNAiMAX Transfection Reagent.
c. 24 hours after that, IL-1 was added for the purpose of inducing expression of a known gene that promotes inflammation, angiogenesis, cartilage destruction, and the like.
d. After a further 24 hours, the effect of suppressing the expression of cartilage catabolic genes (MMP-3 and RUNX2) and inflammation-associated genes (IL-1β, IL-6, and TNF-α) was confirmed by q-PCR, and compared with the group transfected with Negative control miRNA mimic having no target gene.

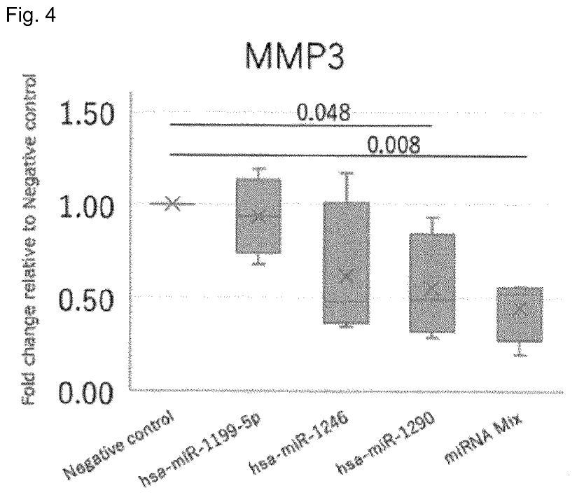

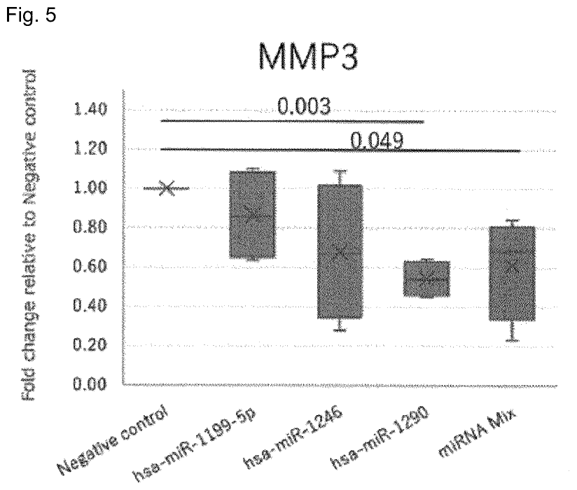

The genes with expression significantly suppressed as compared with the negative control miRNA mimic and the significant probabilities thereof (p values) are shown in Table 3 below. In addition, FIG. 4 shows a graph of the expression level of MMP-3 in SW1353, and FIG. 5 shows a graph of the expression level of MMP-3 in SW982.

TABLE 3

| Target cell type | Gene name | hsa-miR-1199-5p | hsa-miR-1246 | hsa-miR-1290 | miRNA Mix |
|---|---|---|---|---|---|
| SW1353 | MMP-3 | | | 0.048 | 0.008 |
| SW982 | RUNX2 | | 0.001 | 0.008 | 0.009 |
| | MMP-3 | | | 0.003 | 0.049 |
| | IL-1β | 0.009 | 0.002 | | |
| | IL-β | 0.023 | | | |
| | TNF-α | 0.036 | | | |

As shown in Table 3 above, the hsa-miR-1199-5p significantly suppressed the expression of IL-1β, IL-6, and TNF-α in synoviocytes. The hsa-miR-1246 significantly suppressed the expression of IL-1 in synoviocytes. The hsa-miR-1290 significantly suppressed the expression of MMP-3 in chondrocytes and synoviocytes. The hsa-miRs-1246 and 1290 significantly suppressed the expression of RUNX2 in chondrocytes. miRNAMix in which 3 types of miRNAs of hsa-miR-1199-5p, 1246, and 1290 were mixed significantly suppressed the expression of MMP-3 in chondrocytes and synoviocytes and the expression of RUNX2 in chondrocytes.

In addition, the results shown in FIGS. 4 and 5 indicate that the gene silencing for MMP-3 in chondrocytes may be enhanced by combining 3 types of miRNAs of hsa-miR-1199-5p, 1246, and 1290, as compared with the miRNA singly.

The results of the series of experiments described above support the following.

The hsa-miRs-1199-5p and 1246 can suppress the expression of genes involved in the induction of inflammation and genes involved in the induction of cartilage degeneration, and thus can contribute to the suppression of the induction of inflammation and the suppression of the induction of cartilage degeneration in a joint. Therefore, the hsa-miRs-1199-5p and 1246 are effective for treating and/or preventing inflammation in a joint and treating and/or preventing cartilage degeneration in a joint, and are thus effective for treating and/or preventing joint disease.

The hsa-miR-1290 can suppress the expression of genes involved in the induction of cartilage degeneration, and thus can contribute to the suppression of the induction of cartilage degeneration in a joint. Therefore, the hsa-miR-1290 is effective for treating and/or preventing cartilage degeneration in a joint, and thus is effective for treating and/or preventing joint disease.

The combination of the miRNAs of the hsa-miRs-1199-5p, 1246, and 1290 is considered to be effective for treating and/or preventing cartilage degeneration in a joint, and thus is considered to be effective for treating and/or preventing joint disease.

In addition, in the test results of (1-7) (Table 2 and FIG. 3), the correlation coefficients of the hsa-miR-1246 and the hsa-miR-1290 were higher than that of the hsa-miR-1199-5p. From this result, it is considered that the hsa-miR-1246 and/or the hsa-miR-1290 show a higher effect for treating and/or preventing joint disease (in particular, cartilage degeneration in a joint).

### Example 2

### (2-1) Experimental Procedure

### (2-1-1) Cell Sheet Culture

Chondrocytes were isolated from polydactyly surgical waste tissue (4 males and 4 females, age of 8 to 17 months, average age of 12.6 months) collected from 8 donors (donors 1 to 8) to prepare a first passage cell stock.
At the time of preparing cell sheet (PD sheet), cell stocks were thawed and a third passage cell was seeded at a density of 1×10⁴/cm² in a temperature-responsive UpCell insert (CellSeed). Cell sheets were cultured under the conditions of PD-A Exo. Specifically, a DMEM-F12 medium containing 20% FBS, 1% Anti-Biotics, and 100 μg/mL Ascorbic Acid was used as a culture medium, a temperature-responsive culture insert (UpCell (registered trademark) insert, manufactured by CellSeed Inc.) was used as a culture vessel, and static culture was performed at a culture temperature.
Medium exchange was performed every 3 to 4 days.

### (2-1-2) Collection of Culture Supernatant and Cell Sheet Sample

The cell sheet sample was sampled 2 weeks after the start of culture and used for the experiment.
The culture supernatant sample was collected after a lapse of 72 hours from the 11th day from the start of culture.
Centrifugation was performed at 2000 g, 10 minutes, and 4° C., and cell vesicles and cell debris deviating from the exosome fraction were removed through a 0.22 μm filter. A sample was stored at −80° C.
For the purpose of eliminating the influence of the Exosomal miRNA derived from FBS present in the culture solution, a medium containing 20% FBS was used as a negative control.

### (2-1-3) Test for Confirming Effectiveness of PD Sheet Using In Vitro Evaluation System (PD Sheet-Derived Pellet)

The completed PD sheet was collected from the UpCell insert, cultured in suspension in a low-adhesion 6-well plate, and evaluated in two items of a: gene expression analysis, and b: histological evaluation.
   a. Gene Expression Analysis:
The PD sheet-derived pellet on the 7th day of suspension culture was collected in 1 mL of TRIzol reagent (Qiagen), crushed using stainless steel beads and SHAKE Master Neo (BMS), and stored at −80° C. until used for the experiment.
Total RNA was extracted using the thawed sample and using RNeasy Mini Kit (Qiagen), and cDNA was synthesized using QuantiTect Reverse Transcription Kit (Qiagen).
Gene expression of Collagen Type 1 (COL1A1) and Type2 (COL2A1) was confirmed by q-PCR method using Power SYBR GreenMaster Mix (Thermo Fisher Scientific).
Hyaline cartilage forming ability of each donor was evaluated using the gene expression ratio of COL2A1 and COL1A1 (COL2A1/COL1A1 value).
   b. Histological Evaluation:
The PD sheet-derived pellet on the 28th day of suspension culture was fixed with formalin and then embedded in paraffin to prepare a block.
A thin sliced section having a thickness of 3 μm was prepared with a microtome, and subjected to Safranin O (Safranin O/Fast Green/Hematoxylin) and Toluidine Blue staining according to a conventional method to evaluate whether the PD sheet itself has differentiation potency into cartilage.
   (2-1-4) Small RNA-Sequence and Validation Test by q-PCR

The following tests were performed on the PD sheets with the effectiveness evaluated in the above (2-1-3).
For the culture supernatant sample, RNA was extracted from the exosome using exoRNeasy Serum/Plasma Midi Kit (QIAGEN).
For cell sheet samples, RNA was extracted using the TRIzol reagent (Thermo Scientific) according to the manufacturer's protocol.
Two comparative experiments of Group A and Group B were performed by small RNA-sequence, and among the extracted miRNAs, miRNAs commonly highly expressed in both groups were extracted.

### Group A:

Extraction of significantly up-regulated exosomal miRNAs in the PD sheets with high effectiveness compared to the PD sheets with low effectiveness (p<0.05, Log FC>1)

### Group B:

Extraction of miRNAs significantly enriched as exosomal miRNAs in the exosome released by the cell sheet compared to miRNAs expressed in cells constituting the sheet with high effectiveness (p<0.05, Log FC>1)
Sequence libraries were adjusted using the QIAseq miRNA Library Kit (QIAGEN) and QIAseq miRNA NGS 96 Index IL (QIAGEN). Sequence analysis was performed by a single-ended method (75 bp) using the NextSeq500 (ILLUMINA) system.
109 miRNAs with the expression significantly upregulated in common to Groups A and B were extracted, and validation tests by q-PCR were performed on 15 miRNAs in which Log 2FC values were ranked high.
A q-PCR reaction was performed using cDNA synthesized from the RNA sample and using miRCURY LNA RT Kit (QIAGEN) and miRCURY probe PCRKit (QIAGEN).

### (2-1-5) RNA-Seq Data Analysis

Using sequence reads of small RNA-seq, alignment with miRBase 21v was performed at GeneGlobe: Data Analysis Center. After normalization by the TMM method, hierarchical clustering analysis, principal ingredient analysis, and variation gene analysis (p<0.05, Log 2FC>1) of all samples on miRNA expression were performed. At this time, miRNAs having a read count (normalized) of more than 3.0 in negative control were excluded from the subsequent analysis targets.
   (2-1-6) Verification of Silencing of miRNA Cartilage Catabolism and Inflammation-Associated Genes

The functions of hsa-miR-141-5p and 4700-5p on cartilage catabolism and inflammation-associated gene in target cells were investigated.
Each miRNA was transfected into Human Bone chondrosarcoma cell line SW1353 (n=3) or Human Synovial sarcoma cell line SW982 (n=3).
24 hours after cell seeding, hsa-miR-141-5p, 4700-5pmimic (mirVana (registered trademark) miRNA Mimics) was transfected using Lipofectamine (registered trademark) RNAiMAX Transfection Reagent.
24 hours after that, IL-1b was added for the purpose of inducing expression of a known gene that promotes inflammation, angiogenesis, cartilage degradation, and the like.
After a further 24 hours, the effect of suppressing expression of cartilage catabolism and inflammation-associated genes was confirmed by q-PCR, and compared with a group in which negative control miRNA mimic having no target gene was transfected.

From the experimental results described above, it was expected that 4700-5p had a higher therapeutic effect on joint disease among the two identified miRNAs (refer to experimental results (2-2-4) and (2-2-5)). Therefore, the following experiments were performed only for 4700-5p.

### (2-1-7) RIP-Assay

For Human Bone chondrosarcoma cell line SW1353 (n=3) or Human Synovial sarcoma cell line SW982 (n=3), hsa-miR-4700-5p or negative control miRNA mimic having no target gene was transfected. After 24 hours, the cells were collected, and RNA binding protein Assay was performed using RIP-Assay Kit for microRNA (MEDICAL & BIOLOGICAL LABORATORIES CO., LTD.) and Anti-EIF2C2 (AGO2) (Human) mAb (MEDICAL & BIOLOGICAL LABORATORIES CO., LTD.), and mRNA paired with miRNA via AGO2 that is an RNA-binding protein was selectively collected, and total RNA was extracted.
Microarray analysis was performed using Low Input Quick Amp Labeling Kit and SurePrint G3 Human GE Microarray 8×60 K Ver3.0 (Design ID: 072363).
Compared to cells transfected with negative control, significantly upregulated genes were narrowed in cells transfected with miRNA-4700-5p.
In RIP-Assay, enrichment analysis was performed using Panther Classification System v. 16.0 on a gene group upregulated 2 times or more in cells transfected with miRNA-4700-5p, and a pathway in which the gene candidate group was significantly enriched was investigated. Among the genes assigned to these pathways, genes upregulated 5 times or more in cells transfected with miRNA-4700-5p were picked up by RIP-Assay, and the function of genes involved in joint disease was investigated.

### (2-2) Experimental Results

### (2-2-1.) Results of the Test for Confirming Effectiveness of PD Sheet

a. Gene Expression Analysis Result

COL2A1 is known as a marker gene of hyaline cartilage constituting articular cartilage, and COL1A1 is known as a marker gene of fibrocartilage inferior in quality to the hyaline cartilage. As a result of comparing the gene expression levels relative to the COL2A1/COL1A1 value of donor-1 in which the expression level of COL2A1 was the lowest among the 8 donors, compared to donor 1, the COL2A1/COL1A1 values in donor-2, 3, and 4 were 7 to 11 times larger, whereas the COL2A1/COL1A1 values in donor-5, 6, 7, and 8 were 86 to 4091 times larger, among the 8 donors. That is, it was suggested that the donor-5 to 7 have higher hyaline cartilage forming ability than the donor-1 to 4.

Among them, as a result of confirming the correlation between the ICRS score value and the COL2A1/COL1A1 value for 6 donors (Toyoda et al., 2019.) of donors 1, 2, 3, 5, 6, and 7 in which data of scores of histological evaluation (International Cartilage Repair Societyscore; ICRS score) obtained in experiments of the in vivo evaluation system that we have accumulated so far, the determination coefficient (R²) was 0.794, and a high correlation was observed.

b. Histological Evaluation Result

From the result of gene expression analysis, it was shown that the donor-1-to-4-derived pellet in which the COL2A1/COL1A1 value was high, that is, the hyaline cartilage forming ability was expected to be high had a strong dyeability of Safranin O and a metachromatic property of Toluidine Blue, and had a high hyaline cartilage differentiation ability. On the other hand, in the donor donor-5-to-7-derived pellet expected to have low hyaline cartilage forming ability, weak dyeability of Safranin O and weak metachromatic property of Toluidine Blue were confirmed, and it was suggested that differentiation in the hyaline cartilage direction hardly occurred.

From the results of a. and b. above, 4 donors of donor-1 to 4 were selected as the PD sheets with high effectiveness, and 4 donors of donor-5 to 8 were selected as the PD sheets with low effectiveness.

### (2-2-2) Small RNA-Sequence Analysis Result

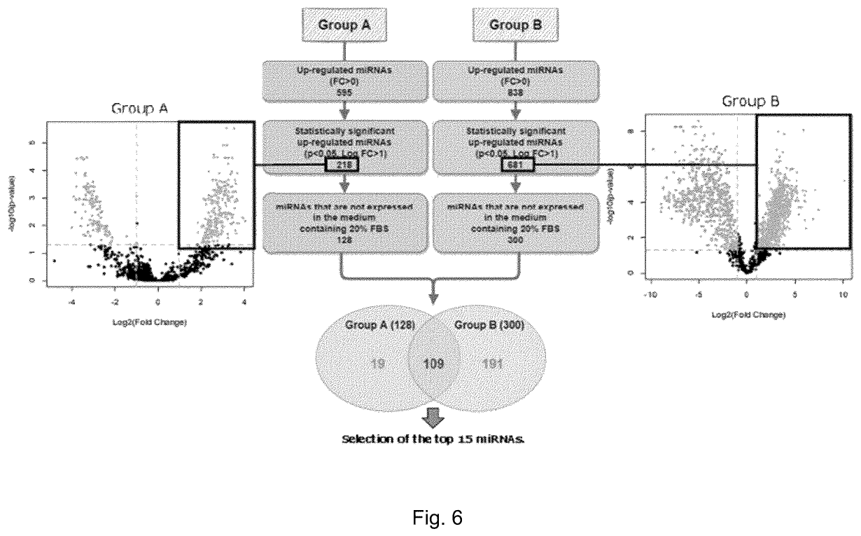

As a result of variation gene analysis, in the comparison of Group A, among the 595 miRNAs that were upregulated, the number of miRNAs with the expression significantly varied was 218. The number of miRNAs in which the expression was considered to have truly varied by excluding the influence of negative control was 128 (FIG. 6).

In the comparison of Group B, among the 838 miRNAs that were upregulated, the number of miRNAs with the expression significantly varied was 681. The number of miRNAs in which the expression was considered to have truly varied by excluding the influence of negative control was 300 (FIG. 6).

(2-2-3) Results of Validation Test by q-PCR

A validation test by q-PCR was performed on 15 miRNAs having the highest value of Log 2FC among 109 miRNAs in which the expression was significantly upregulated in common to Groups A and B. In comparison of both Group A and B, there were 2 miRNAs with significant expression variation of p<0.05, Log 2FC>1: hsa-miR-141-5p and hsa-miR-4700-5p (Table 4). In order to compare the tendency with the data of small RNA-seq, the correlation between the read count data and the −ΔCT value of q-PCR was confirmed, and a high correlation of the correlation coefficient (R)=0.6 to 0.74 was observed in both comparisons of Groups A and B in any miRNA (Table 4).

TABLE 4
Table: Confirmation of correlation between
RNA-seq data and q-PCR validation test

| | | miRNA-seq data | | q-PCR data | | Correlation |
|---|---|---|---|---|---|---|
| Comparison | | p-value | Log | | Log | coefficient |
| group | Name | (Corr) | FC | p-value | FC | (R) |
| Group A | hsa-miR-141-5p | 0.0085 | 3.7 | 0.0265 | 2.7 | 0.60 |
| Group B | | 0.0015 | 3.7 | 0.0023 | 5.4 | 0.60 |
| Group A | hsa-miR-4700-5p | 0.0001 | 3.3 | 0.0459 | 5.0 | 0.74 |
| Group B | | 0.0000 | 3.3 | 0.0068 | 10.3 | 0.74 |

Two miRNAs, hsa-miR-141-5p and hsa-miR-4700-5p that are characteristically highly expressed within the highly effective PD sheet exosome were identified.

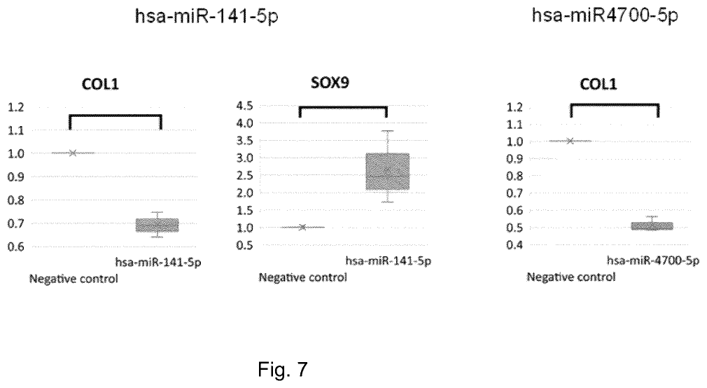

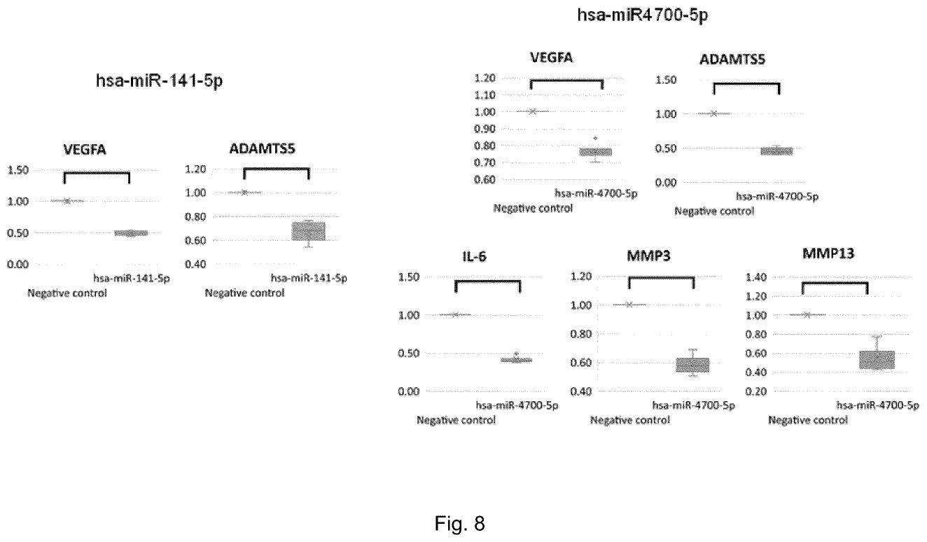

(2-2-4) Verification Results of Silencing miRNA Cartilage Catabolism and Inflammation-Associated Genes (FIGS. 7 and 8)

For gene expression in chondrocytes, miR-141-5p and 4700-5p suppressed Col1 expression and miR-141-5p enhanced Sox9 gene expression.

For the gene expression in synoviocytes, miR-141-5p and 4700-5p suppressed the gene expression of ADAMTS5 and VEGFA, and miR-4700-5p also suppressed the expression of matrix metalloproteases such as MMP3 and MMP13 and IL-6 as an inflammatory system marker gene.

From these results, it is found that the target gene of miRNA-141-5p is likely to be upstream gene suppression that induces or promotes CA pathology and cartilage degeneration and controls angiogenesis, and as a result, it is possible to bring about suppression of gene expression of COL1, ADAMTS5, and VEGFA, enhancement of gene expression of SOX9, and contribute to suppression of induction of cartilage degeneration.

In addition, the target gene of miRNA-4700-5p is likely to be upstream gene suppression that induces an inflammatory reaction, induces or promotes OA pathology or cartilage degeneration, and controls angiogenesis, and as a result, it may lead to suppression of gene expression of COL1, MMP3, MMP13, ADAMTS5, VEGFA, and IL-6, and contribute to improvement of the intraarticular environment (suppression of inflammation, suppression of angiogenesis, and the like) and cartilage repair.

### (2-2-5) Search for Target Gene by RIP-Assay

As a result of extracting genes significantly upregulated in cells transfected with miRNA-4700-5p as compared with cells transfected with negative control by RIP-Assay, 246 genes were extracted with FC>2, 21 genes with FC>5, 1 gene with FC>10 in Human Bone chondrosarcoma cell line SW1353, 598 genes with FC>2, 68 genes with FC>5, and 11 genes with FC>10 in human synovial sarcoma cell line SW982 (n=3).

As a result of performing enrichment analysis using the Panther Classification System v. 16.0 on a gene group (246 cells in Human Bone chondrosarcoma cell line SW1353, 598 cells in Human Synovial sarcoma cell line SW982 (n=3)) upregulated 2 times or more by cells transfected with miRNA-4700-5p, no pathways were detected that were significantly enriched in the gene candidate group in Human Bone chondrosarcoma cell line SW1353, whereas in Human Synovial sarcoma cell line SW982, 13 types of pathways were detected, including Angiogenesis (P00005), Interleukin signaling pathway (P00036), VEGF signaling pathway (P00056), Inflammation mediated by chemokine and cytokine signaling pathway (P00031), and the like.

TABLE 5
Table: Pathway of enriched miRNA paired with hsa-
miR-4700-5p among genes expressed in synoviocytes

| PANTHER Pathways | fold Enrichment | p-value |
|---|---|---|
| Angiogenesis (P00005) | 2.94 | 0.001 |
| Ras Pathway (P04393) | 4.31 | 0.001 |
| CCKR signaling map (P06959) | 2.6 | 0.005 |
| Interleukin signaling pathway (P00036) | 3.22 | 0.008 |
| VEGF signaling pathway (P00056) | 3.61 | 0.008 |
| Plasminogen activating cascade (P00050) | 7.22 | 0.011 |
| Cell cycle (P00013) | 5.34 | 0.023 |
| Axon guidance mediated by | 4.91 | 0.028 |
| Slit/Robo (P00008) | | |
| Notch signaling pathway (P00045) | 3.64 | 0.029 |
| PDGF signaling pathway (P00047) | 2.26 | 0.031 |
| TCA cycle (P00051) | 7.44 | 0.037 |
| Inflammation mediated by chemokine and | 1.88 | 0.042 |
| cytokine signaling pathwaw (P00031) | | |
| FGF signaling pathway (P00021) | 2.26 | 0.042 |

Among the genes assigned to 13 types of pathways that were significantly detected by Human Synovial sarcoma cell line SW982, 4 genes were upregulated 5 times or more in the cells transfected with miRNA-4700-5p in RIP-Assay (PDHA1, RAB11B, IL-6R, PRKG1). Among them, IL-6R is a receptor of IL-6 that has been reported to play an important role in processes such as inflammation, immune reaction, and hematopoiesis, and directly inhibit differentiation of chondroprogenitor cells. Increased IL-6 production is known to be involved in the mechanisms of development of many inflammatory diseases. The humanized anti-human IL-6R monoclonal antibody “tocilizumab” suppresses bone destruction and cartilage destruction in a patient with rheumatoid arthritis. In addition, it has been reported that PRKG1 is involved in bone regeneration, and it has been reported that in KO mice of PRKG1 gene, gene expression of VEGF and BMP2/4 is reduced and bone regeneration is suppressed in osteoblasts.

The function of the gene paired with miRNA-4700-5p via AGO2 protein was a gene group that controls induction of inflammatory reaction, induction and promotion of CA pathology and cartilage degeneration, and angiogenesis.

As is apparent from the above results, miR-4700-5p is an Exosomal miRNA characteristically highly expressed in Exosome released by a highly effective PD sheet. This targets a gene group including IL-6R and PRKG1 belonging to pathway involved in angiogenesis, inflammation, and degeneration of cartilage in synoviocytes, suppresses expression of MMP13, MMP3, ADAMTS5, VEGF, and the like, which are downstream genes deeply involved in the pathology of OAK, and may contribute to improvement of the intraarticular environment and cartilage repair.

1. A method for treating and/or preventing a joint disease, comprising the step of administering an effective amount of at least one miRNA selected from the group consisting of hsa-miR-1199-5p, hsa-miR-1246, hsa-miR-1290, hsa-miR-141-5p and hsa-miR-4700-5p to a subject in need thereof.

2. A method for treating and/or preventing a joint disease, comprising the step of administering an effective amount of a miRNA,
wherein the miRNA satisfies one or both of requirements (i) and (ii) below:
(i) a miRNA that suppresses an expression level of at least one factor selected from the group consisting of MMP-3, IL-1β, IL-6, TNF-α, MMP-13, ADAMTS5, and VEGFA in a case of being transfected into a Human Synovial sarcoma cell line as compared with a Human Synovial sarcoma cell line transfected with a negative control miRNA mimic having no target gene, and
(ii) a miRNA that suppresses an expression level of at least one factor selected from the group consisting of MMP-3 and RUNX2 in a case of being transfected into a Human Bone chondrosarcoma cell line as compared with a Human Bone chondrosarcoma cell line transfected with a negative control miRNA mimic having no target gene.

3. The method according to claim 1, wherein the miRNA is included in an exosome.

4. The method according to claim 3, wherein the exosome is obtained from a cell derived from a joint tissue.

5. The method according to claim 4, wherein the cell derived from the joint tissue is obtained from a cell culture derived from a joint tissue.

6. The method according to claim 1, wherein the treating and/or preventing a joint disease is treating and/or preventing inflammation in a joint.

7. The method according to claim 1, wherein
the treating and/or preventing the joint disease is treating and/or preventing inflammation in a joint, and
the active ingredient is at least one selected from the group consisting of the hsa-miR-1199-5p, hsa-miR-1246, and hsa-miR-4700-5p.

8. The method according to claim 1, wherein the treating and/or preventing the joint disease is treating and/or preventing cartilage degeneration in a joint.

9. The method according to claim 1, wherein
the treating and/or preventing the joint disease is treating and/or preventing cartilage degeneration in a joint, and
the active ingredient is at least one selected from the group consisting of hsa-miR-1199-5p, hsa-miR-1246, hsa-miR-1290, hsa-miR-141-5p, and hsa-miR-4700-5p.

10. The method according to claim 8, wherein the treating and/or preventing cartilage degeneration in the joint is treating and/or preventing cartilage damage in a joint.

11. The method according to claim 1, wherein the miRNA is administered by injection.

12. The method according to claim 1, wherein the miRNA is included in a syringe barrel.

13-17. (canceled)

18. A marker for determining whether or not a cell aggregate is effective for treating and/or preventing a joint disease, the marker being made of at least one miRNA selected from the group consisting of hsa-miR-1199-5p, hsa-miR-1246, hsa-miR-1290, hsa-miR-141-5p, and hsa-miR-4700-5p.

19. A marker for determining whether or not a cell aggregate is effective for treating and/or preventing a joint disease, the marker being made of a miRNA,
wherein the miRNA satisfies one or both of requirements (i) and (ii) below:
(i) a miRNA that suppresses an expression level of at least one factor selected from the group consisting of MMP-3, IL-1β, IL-6, TNF-α, MMP-13, ADAMTS5, and VEGFA in a case of being transfected into a Human Synovial sarcoma cell line as compared with a Human Synovial sarcoma cell line transfected with a negative control miRNA mimic having no target gene, and
(ii) a miRNA that suppresses an expression level of at least one factor selected from the group consisting of MMP-3 and RUNX2 in a case of being transfected into a Human Bone chondrosarcoma cell line as compared with a Human Bone chondrosarcoma cell line transfected with a negative control miRNA mimic having no target gene.

20. A method for determining whether or not it is effective for treating and/or preventing a joint disease using, as an index, a miRNA in an exosome released from a cell derived from a joint tissue to be targeted,
wherein the miRNA is at least one selected from the group consisting of hsa-miR-1199-5p, hsa-miR-1246, hsa-miR-1290, hsa-miR-141-5p, and hsa-miR-4700-5p.

21. A method for determining whether or not it is effective for treating and/or preventing a joint disease using, as an index, a miRNA in an exosome released from a cell derived from a joint tissue to be targeted,
wherein the miRNA satisfies one or both of requirements (i) and (ii) below:
(i) a miRNA that suppresses an expression level of at least one factor selected from the group consisting of MMP-3, IL-10, IL-6, TNF-α, MMP-13, ADAMTS5, and VEGFA in a case of being transfected into a Human Synovial sarcoma cell line as compared with a Human Synovial sarcoma cell line transfected with a negative control miRNA mimic having no target gene, and
(ii) a miRNA that suppresses an expression level of at least one factor selected from the group consisting of MMP-3 and RUNX2 in a case of being transfected into a Human Bone chondrosarcoma cell line as compared with a Human Bone chondrosarcoma cell line transfected with a negative control miRNA mimic having no target gene.

22. A method for producing an exosome including at least one miRNA selected from the group consisting of hsa-miR-1199-5p, hsa-miR-1246, hsa-miR-1290, hsa-miR-141-5p, and hsa-miR-4700-5p, the method comprising a step of culturing a cell including at least one miRNA selected from the group consisting of hsa-miR-1199-5p, hsa-miR-1246, hsa-miR-1290, hsa-miR-141-5p, and hsa-miR-4700-5p.
